(19) United States
(12) Patent Application Publication
(10) Pub. No.: US 2010/0256207 A1
(43) Pub. Date: Oct. 7, 2010

(54) AMORPHOUS OLMESARTAN MEDOXOMIL

(76) Inventor: Axel Becker, Hertfordshire (GB)

Correspondence Address: FULBRIGHT & JAWORSKI L.L.P.;Attn: MN IP Docket, 600 Congress Avenue, Suite 2400, Austin, TX, 78701, US

(21) Appl. No.: 12/602,958

(22) PCT Filed: Jun. 6, 2008
(86) PCT No.: PCT/GB08/50419

§ 371 Date: Jun. 2, 2010

(30) Foreign Application Priority Data
Jun. 7, 2007 (GB) 0710905.1

(51) Int. Cl. A61K 31/41; C07D 257/04; A61P 9/12

(52) U.S. Cl. 514/382; 548/253

(57) ABSTRACT

The present invention is directed towards amorphous olmesartan medoxomil, to methods for preparing the compound, to compositions comprising the compound, and to the use of said compound and compositions for the treatment or prevention of an angiotensin II receptor mediated disorder, in particular hypertension.

### CROSS-REFERENCE TO RELATED APPLICATION(S)

This application is a Section 371 National Stage Application of International No. PCT/GB2008/050419, filed 6 Jun. 2008 and published as WO 2008/149160 A1 on 6 Jun. 2008, which claims priority from the Great Britain Application 0710905.1, filed 7 Jun. 2007, the contents of which are incorporated herein in their entirety for all purposes.

### FIELD OF THE INVENTION

The present invention is directed towards amorphous olmesartan medoxomil, to methods for preparing the compound, to compositions comprising the compound, and to the use of said compound and compositions for the treatment or prevention of an angiotensin II receptor mediated disorder, in particular hypertension.

### BACKGROUND OF THE INVENTION

Olmesartan medoxomil is described chemically as 2,3-dihydroxy-2-butenyl 4-(1-hydroxy-1-methylethyl)-2-propyl-1-[p-(o-tetrazol-5-yl-phenyl)benzyl]imidazole-5-carboxylate cyclic 2,3-carbonate and has the structural formula (I):

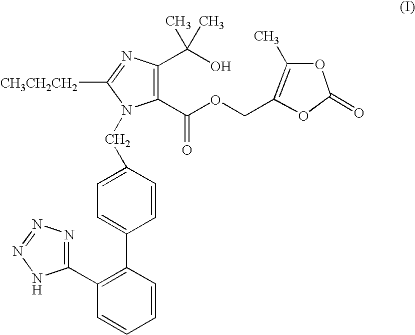

Olmesartan medoxomil is an anti-hypertensive pro-drug ester that is hydrolyzed to olmesartan during absorption from the gastrointestinal tract. It is a selective AT1 subtype angiotensin II receptor antagonist and blocks the vasoconstrictor effects of angiotensin II by selectively blocking the binding of angiotensin II to the AT1 receptor in vascular smooth muscle. Olmesartan medoxomil is indicated for the treatment of hypertension and is commercially sold under the trade name Benicar®.

EP 0503785 describes olmesartan medoxomil and discloses in example 61(b) a process for its preparation. The disclosed process results in a crystalline form characterized in The Annual Report of Sankyo Research Laboratories, vol. 55, 2003. There is no mention of an amorphous form of olmesartan medoxomil.

U.S. Patent Publication No. 2006/0281800 discloses form G olmesartan medoxomil as another crystalline form for use by the skilled person. However, performance data, for example, the results of stability or solubility testing, are not included in the disclosure. Again, there is no mention of an amorphous form of olmesartan medoxomil.

Olmesartan medoxomil has a very low aqueous solubility. This can be problematic when developing pharmaceutical products, as solubility of the active pharmaceutical ingredient (API) is a key parameter to be considered. Prior art solutions to the problem of APIs with low aqueous solubility in general include the development of crystalline forms and amorphous forms having increased dissolution profiles.

Therefore a need exists to overcome one or more of the disadvantages noted above.

### SUMMARY OF THE INVENTION

Due to the low aqueous solubility of olmesartan medoxomil, there is a need for alternative forms of this compound potentially having increased solubility for use in the development of pharmaceutical products. There is also a need for stable forms of olmesartan medoxomil suitable for pharmaceutical development.

The present invention provides a novel amorphous form of olmesartan medoxomil, processes for preparing it, and pharmaceutical formulations comprising it.

Accordingly, a first aspect of the present invention provides amorphous olmesartan medoxomil.

It is known in the art that amorphous forms of active pharmaceutical ingredients (APIs) generally exhibit increased solubility over the corresponding crystalline forms, as lattice energy does not have to be overcome in order to dissolve the solid state structure as is the case for crystalline forms. However, amorphous forms of olmesartan medoxomil have not been described previously in the prior art.

In a second aspect according to the invention there is provided amorphous olmesartan medoxomil characterized by an X-ray powder diffraction pattern substantially as shown in FIG. 1.

A third aspect of the present invention provides amorphous olmesartan medoxomil characterized by a differential scanning calorimetry thermogram substantially as shown in FIG. 2.

It is well known in the art that an API with low hygroscopicity is advantageous over more hygroscopic forms. Hygroscopicity is the property of some compounds to readily absorb water from the surrounding atmosphere. This is generally seen as a negative property, as hygroscopic materials are generally more difficult to handle. Such materials may require additional processing steps to ensure exclusion of moisture from the manufacturing process, and the final dosage form may also require high moisture barrier packages that maintain the integrity and properties of the dosage form. Such measures can increase the cost of manufacturing, processing and packaging of a pharmaceutical product. The exclusion of water may be necessary in the particular case of olmesartan medoxomil, as it is a pro-drug ester which could hydrolyze/degrade to the acid, active metabolite component. The presence of an acid group on the active metabolite renders said metabolite non-permeable and consequently unavailable to the body. Thus, controlling moisture is important and providing a non-hygroscopic API, which can reduce the cost of additional moisture controlling measures and packaging, is particularly advantageous. The inventors have found that amorphous olmesartan medoxomil according to the invention has surprisingly low hygroscopicity.

Accordingly, there is provided in a fourth aspect of the invention amorphous olmesartan medoxomil substantially free of water. A preferred embodiment provides amorphous olmesartan medoxomil having a water content of less than about 2% by weight, preferably less than about 1% by weight, more preferably less than about 0.5% by weight (as measured by TGA).

A fifth aspect of the present invention provides amorphous olmesartan medoxomil substantially free of crystalline olmesartan medoxomil. The term “substantially free” of crystalline olmesartan medoxomil as used herein means less than about 10% of crystalline olmesartan medoxomil. In one embodiment, the amorphous olmesartan medoxomil according to the invention comprises less than about 10% crystalline olmesartan medoxomil, preferably less than about 5%, more preferably less than about 1%, even more preferably less than about 0.5%, and most preferably less than about 0.1% (as measured by XRPD or DSC).

The amorphous olmesartan medoxomil of the invention possesses good dissolution characteristics and good stability over the time and temperature ranges to which pharmaceutical compositions are generally subjected, both in use and in testing for regulatory approval. Thus the amorphous olmesartan medoxomil is suitable for pharmaceutical formulation as an angiotensin type II receptor antagonist. Thus the amorphous olmesartan medoxomil of the present invention is suitable for use in medicine, preferably for treating or preventing an angiotensin type II receptor mediated disorder such as hypertension.

In a sixth aspect according to the invention there is provided a process for preparing amorphous olmesartan medoxomil, comprising the steps of:

(a) dissolving or suspending olmesartan medoxomil in one or more organic solvent(s); and
(b) isolating amorphous olmesartan medoxomil.

In preferred embodiments, the solvent(s) is/are alcohol solvents, preferably short-chain (C1-C4) alcohols, more preferably methanol, ethanol, anhydrous ethanol or propanol. In alternative embodiments, the solvent(s) is/are nitrile solvents, preferably short-chain (C1-C4) nitriles, particularly preferred is acetonitrile. Preferably, the solvent(s) is/are HPLC-grade.

In preferred embodiments, in step (a) olmesartan medoxomil is dissolved in one or more organic solvent(s). Preferably the solution or suspension obtained in step (a) is subjected to sonication or heating (preferably sonication) to aid the dissolution of the olmesartan medoxomil.

In preferred embodiments of the process, the solution or suspension obtained in step (a) is filtered, preferably through a filter having a pore size of about 0.3-1.0 μm, preferably the pore size is between about 0.4-0.6 μm, and more preferably the pore size is about 0.45 μm.

A preferred embodiment of the sixth aspect provides isolating amorphous olmesartan medoxomil by allowing the solvent to evaporate by one of the methods selected from the group comprising: spray drying, flash drying, heating, evaporation under reduced pressure, and evaporation under ambient conditions (i.e. room temperature and atmospheric pressure). Preferably, the solvent is allowed to evaporate under ambient conditions. In a particularly preferred embodiment, the solvent is allowed to evaporate under ambient conditions, preferably at a temperature of between 20-35° C.

Alternatively, an anti-solvent may be added to the solution or suspension obtained in step (a) to force amorphous olmesartan medoxomil out of solution. Preferably, the anti-solvent is capable of dissolving in the solvent used in step (a). Preferably, the anti-solvent is a liquid. Alternatively still, the solution or suspension obtained in step (a) may be cooled to force amorphous olmesartan medoxomil out of solution. Once the amorphous olmesartan medoxomil has been forced out of solution, it can be isolated by any known means such as filtration.

In a seventh aspect according to the invention, a pharmaceutical composition is provided comprising a therapeutically or prophylactically effective amount of amorphous olmesartan medoxomil according to all aspects and embodiments of the invention and at least one pharmaceutically acceptable excipient.

An eighth aspect provides a method of treating or preventing an angiotensin type II receptor mediated disorder, comprising administering to a subject in need of such treatment or prevention, a therapeutically or prophylactically effective amount of amorphous olmesartan medoxomil according to the invention. In a particularly preferred embodiment of the invention, the disorder is hypertension.

A ninth aspect provides a use of amorphous olmesartan medoxomil according to the invention in the manufacture of a medicament for the treatment or prevention of an angiotensin type II receptor mediated disorder. In a particularly preferred embodiment of the invention, the disorder is hypertension.

### BRIEF DESCRIPTION OF THE DRAWINGS

FIG. 1: X-Ray Powder Diffraction (XRPD) pattern of amorphous olmesartan medoxomil according to the invention;

FIG. 2: Differential Scanning Calorimetry (DSC) heating trace of amorphous olmesartan medoxomil;

FIG. 3: Thermo-Gravimetric Analysis (TGA) heating trace of amorphous olmesartan medoxomil.

### DETAILED DESCRIPTION OF THE INVENTION

The present invention relates to amorphous olmesartan medoxomil. Such a compound has not previously been described in the prior art.

The inventors have found that it is particularly difficult to prepare amorphous olmesartan medoxomil. Quench-cooling of the melt, which can be considered the simplest way to obtain analytical amounts of amorphous material, is not possible with olmesartan medoxomil, because the melting process is inherently accompanied by degradation processes.

Accordingly, the amorphous olmesartan medoxomil of the invention may be prepared in one embodiment by dissolving olmesartan medoxomil in an organic solvent. It has been found by the inventors that preferably the organic solvent is methanol, ethanol or acetonitrile. Of course, it will be understood that a number of further organic solvents may be utilised.

In a further embodiment of the process, the olmesartan medoxomil is completely dissolved. This can be achieved by any means known in the art, but particularly preferred is exposing the solution to ultrasonication. Further embodiments comprise sonicating the solution at room temperature, which the skilled person would assume to be about 20-25° C., of course minor adjustments above or below this range are incorporated in the scope of this embodiment. In a further embodiment, the sonication is continued until a clear solution is obtained indicating that all the olmesartan medoxomil has dissolved, preferably this lasts for about 5 minutes. Other means for aiding in the dissolution of the olmesartan medoxomil may comprise heating, of course, the skilled person will understand that the heating will be performed in a way that prevents any possible degradation.

Further embodiments of the process comprise filtering the solution to remove any particulate matter. Such matter may act as seeds and promote the formation of crystalline forms of olmesartan medoxomil in the solution. Preferably, the solution is filtered through a filter which preferably has a pore size of between 0.1 and 1 μm. A 0.45 μm filter is particularly preferred.

Once the solution has been prepared as described above, the solvent in preferred embodiments is allowed to evaporate. Preferably, evaporation is not forced in that heating is not required, however, it has been shown in alternative embodiments by the inventors that vacuum evaporation may be employed. Of course, there may be alternative embodiments known to the skilled person that facilitate the isolation of amorphous olmesartan medoxomil according to the invention. It is envisaged that these fall within the scope of the invention as detailed in the appended claims and described herein. Upon full evaporation of the relevant solvent, amorphous olmesartan medoxomil is obtained.

Polymorphism is the occurrence of different crystalline and amorphous forms of a single compound and it is a property of some compounds and complexes. Polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-ray diffraction peaks. Since the solubility of each polymorph may vary, identifying the existence of pharmaceutical polymorphs is essential for providing pharmaceuticals with predicable solubility profiles. Polymorphic forms of a compound can be distinguished in a laboratory by X-ray diffraction spectroscopy and by other methods such as infrared spectrometry. Additionally, polymorphic forms of the same drug substance or active pharmaceutical ingredient can be administered by themselves or formulated as a drug product (also known as the final or finished dosage form), and are well known in the pharmaceutical art to affect, for example, the solubility, stability, flowability, tractability and compressibility of the drug substance and the safety and efficacy of drug products.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic. It has now been surprisingly found that an amorphous form of olmesartan medoxomil exists.

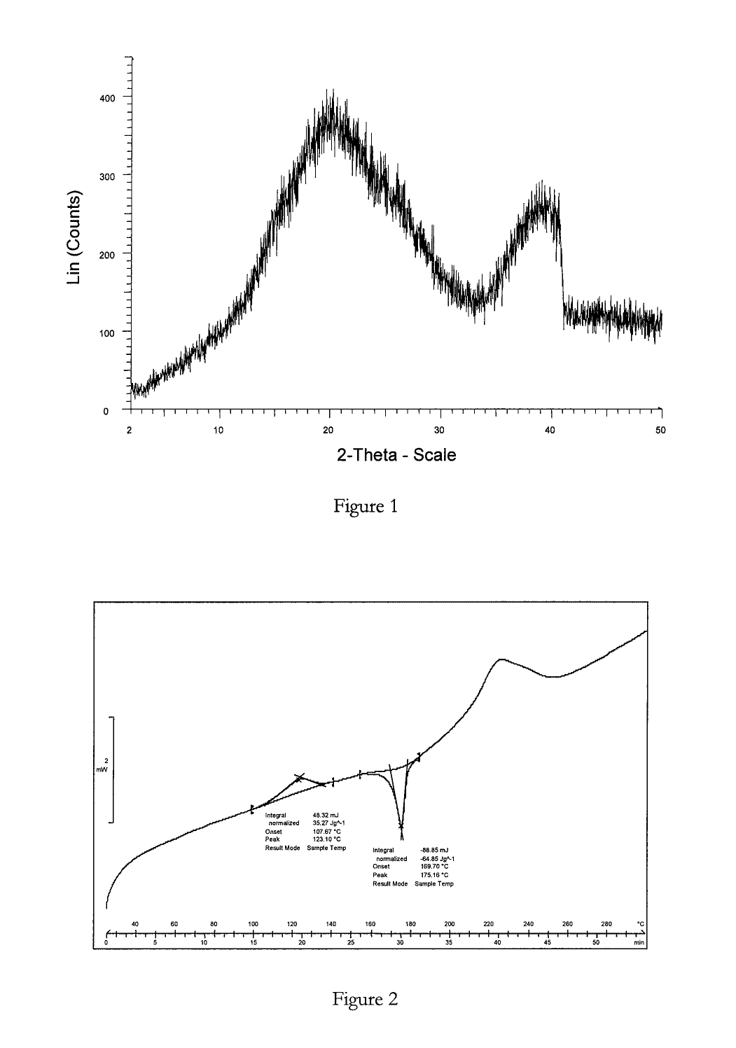

Analysis of the powder obtained by the process of the present invention as described above and in the following examples by XRPD techniques resulted in the trace shown in FIG. 1. It can be seen that there are two humps. The second, in the range 35-40°2θ, is due to the XRPD sample holder and can thus be disregarded. Notwithstanding the second hump, FIG. 1 displays a typical trace characteristic of an amorphous sample. There are none of the characteristic peaks or troughs associated with a sample having a crystalline structure.

The X-ray powder diffraction data was obtained by methods known in the art using a Bruker D8 Advance Powder Diffractometer with scintillation detector under the following parameters:

Reflection mode

Cu K\acute{α} radiation (1.5406 Å)

Scanning range: 2-50°2θ

Step size: 0.02°2θ

Time per step: 2 s

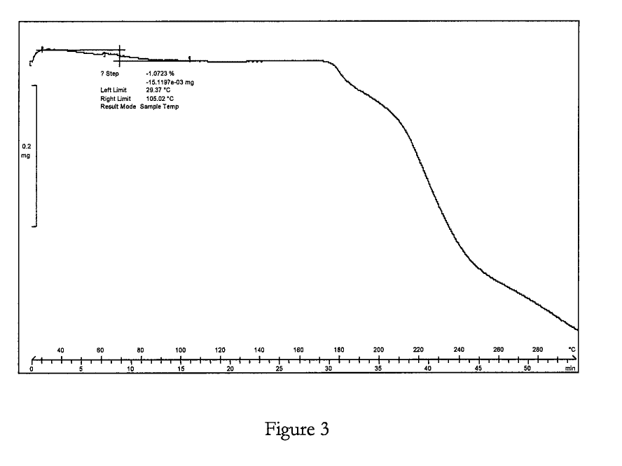

The powder obtained by the process according to the invention as described above and in the following examples was also subjected to Differential Scanning Calorimetry (DSC). The resulting trace is shown in FIG. 2. It will be apparent to one skilled in the art that the exothermic event at 100-140° C. is a heating-induced recrystallisation of the amorphous form to the crystalline prior art form. This is confirmed by XRPD data and confirms that the chemical entity of the initial amorphous material does represent olmesartan medoxomil. Further, it also confirms that the amorphous olmesartan medoxomil according to the above described embodiments of the invention is sufficiently kinetically stable or metastable, as no conversion to other polymorphic forms occurs below 100° C.

The DSC thermal analysis data was obtained using a Mettler-Toledo DSC821e apparatus under the following parameters:

Temperature profile: 25-300° C.@5° C./min

Nitrogen purge gas, 50 ml/min

Aluminium pan, 40 ml, pierced prior to scan

The powder obtained by the process according to the invention as described above and in the following examples was also subjected to Thermo-Gravimetric Analysis (TGA). An exemplary TGA trace is shown in FIG. 3.

The inventors have also found that in certain preferred embodiments of the invention, the amorphous olmesartan medoxomil according to the invention is substantially non-hygroscopic, consequently there is provided amorphous olmesartan medoxomil comprising less than 2% water by weight. The water content was determined by weight loss in the TGA data at 25-105° C.

The TGA analysis data was obtained using a Mettler-Toledo TGA851e apparatus under the following parameters:

Temperature profile: 25-300° C.@5° C./min

Nitrogen purge gas, 50 ml/min

Aluminium pan, 40 ml, pierced prior to scan

Illustrative of the invention is a pharmaceutical composition made by mixing amorphous olmesartan medoxomil according to the invention and a pharmaceutically acceptable carrier. A further embodiment of the invention is a process for making a pharmaceutical composition comprising mixing amorphous olmesartan medoxomil according to the invention and a pharmaceutically acceptable carrier. An example of the invention is a method for the treatment of an angiotensin type II receptor mediated disorder in a subject in need thereof, comprising administering to the subject a therapeutically effective amount of amorphous olmesartan medoxomil according to any of the embodiments of the invention or pharmaceutical compositions described above. Also included in the invention is the use of amorphous olmesartan medoxomil substantially free of crystals, for the preparation of a medicament for treating an angiotensin type II receptor mediated disorder in a subject in need thereof.

Pharmaceutical formulations of the present invention contain amorphous olmesartan medoxomil. It is preferred that the amorphous olmesartan medoxomil is substantially pure, but this is non-limiting to the working of the invention. The amorphous olmesartan medoxomil prepared by the processes of the present invention is ideal for formulation of pharmaceutical products. In addition to the active ingredient(s), the pharmaceutical compositions of the present invention may contain one or more excipients. Excipients are added to the composition for a variety of purposes. Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol®), carboxymethyl cellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminium silicate, maltodextrin, methyl cellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethyl cellulose calcium, carboxymethyl cellulose sodium (e.g. Ac-Di-Sol®, Primellose®), colloidal silicon dioxide, croscarmellose sodium, crospovidone (e.g. Kollidon®, Polyplasdone®), guar gum, magnesium aluminium silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavouring agents and flavour enhancers make the dosage form more palatable to the patient. Common flavouring agents and flavour enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colourant to improve their appearance and/or facilitate patient identification of the product and unit dosage level.

In liquid pharmaceutical compositions of the present invention, olmesartan medoxomil and any other solid excipients are dissolved or suspended in a liquid carrier such as water, vegetable oil, alcohol, polyethylene glycol, propylene glycol or glycerin.

Liquid pharmaceutical compositions may further contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel or organoleptic qualities of the product and/or coat the lining of the gastrointestinal tract. Such agents include acacia, alginic acid, bentonite, carbomer, carboxymethyl cellulose calcium or sodium, cetostearyl alcohol, methyl cellulose, ethyl cellulose, gelatin, guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxytoluene, butylated hydroxyanisole and ethylenediaminetetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts. Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colourant. The active ingredient and excipients may be formulated into compositions and dosage forms according to methods known in the art.

A composition for tableting or capsule filling may be prepared by wet granulation. In wet granulation, some or all of the active ingredient and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the active and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The following paragraphs enumerated consecutively from 1 through 29 provide for various aspects of the present invention. In one embodiment, the present invention provides:

1. Amorphous olmesartan medoxomil.
2. Amorphous olmesartan medoxomil characterized by an X-ray powder diffraction pattern substantially as shown in FIG. 1.
3. Amorphous olmesartan medoxomil characterized by a differential scanning calorimetry thermogram substantially as shown in FIG. 2.
4. Amorphous olmesartan medoxomil substantially free of water.
5. Amorphous olmesartan medoxomil according to paragraph 4, having a water content of less than about 2% by weight.
6. Amorphous olmesartan medoxomil substantially free of crystalline olmesartan medoxomil.
7. Amorphous olmesartan medoxomil according to paragraph 6, comprising less than about 10% crystalline olmesartan medoxomil.
8. Amorphous olmesartan medoxomil according to any one of paragraphs 1-7, for use in medicine.
9. Amorphous olmesartan medoxomil according to any one of paragraphs 1-8, for treating or preventing an angiotensin type II receptor mediated disorder.
10. Amorphous olmesartan medoxomil according to any one of paragraphs 1-9, for treating or preventing hypertension.
11. A process for preparing amorphous olmesartan medoxomil, comprising the steps of:
(a) dissolving or suspending olmesartan medoxomil in one or more organic solvent(s); and
(b) isolating amorphous olmesartan medoxomil.
12. A process according to paragraph 11, wherein the solvent(s) is/are alcohol solvents or nitrile solvents.
13. A process according to paragraph 12, wherein the solvent(s) is/are short-chain alcohol solvents or short-chain nitrile solvents.
14. A process according to paragraph 13, wherein the solvent(s) is/are methanol, ethanol or acetonitrile.
15. A process according to paragraph 14, wherein the solvent(s) is/are anhydrous ethanol.
16. A process according to any one of paragraphs 11-15, wherein the solvent(s) is/are HPLC-grade.
17. A process according to any one of paragraphs 11-16, wherein the solution or suspension obtained in step (a) is subjected to sonication or heating to aid the dissolution of the olmesartan medoxomil.
18. A process according to any one of paragraphs 11-17, wherein the solution or suspension obtained in step (a) is filtered.
19. A process according to paragraph 18, wherein the solution or suspension is filtered through a filter having a pore size of 0.3-1.0 μm.
20. A process according to paragraph 19, wherein the solution or suspension is filtered through a filter having a pore size of 0.4-0.6 μm.
21. A process according to paragraph 20, wherein the solution or suspension is filtered through a filter having a pore size of about 0.45 μm.
22. A process according to any one of paragraphs 11-21, wherein the amorphous olmesartan medoxomil is isolated by allowing the solvent to evaporate.
23. A process according to paragraph 22, wherein the solvent is allowed to evaporate by spray drying, flash drying, heating, evaporation under reduced pressure, or evaporation under ambient conditions.
24. A process according to paragraph 23, wherein the solvent is allowed to evaporate under ambient conditions at a temperature of between 20-35° C.
25. A pharmaceutical composition comprising amorphous olmesartan medoxomil according to any one of paragraphs 1-10, or prepared by a process according to any one of paragraphs 11-24, and at least one pharmaceutically acceptable excipient.
26. A method of treating or preventing an angiotensin type II receptor mediated disorder, comprising administering to a subject in need of such treatment or prevention, a therapeutically or prophylactically effective amount of amorphous olmesartan medoxomil according to any one of paragraphs 1-10, amorphous olmesartan medoxomil prepared by a process according to any one of paragraphs 11-24, or a composition according to paragraph 25.
27. A method according to paragraph 26, wherein the disorder is hypertension.
28. Use of amorphous olmesartan medoxomil according to any one of paragraphs 1-10, or use of amorphous olmesartan medoxomil prepared by a process according to any one of paragraphs 11-24, or use of a composition according to paragraph 25, in the manufacture of a medicament for the treatment or prevention of an angiotensin type II receptor mediated disorder.
29. A use according to paragraph 28, wherein the disorder is hypertension.

The invention is illustrated in more detail by the following non-limiting examples.

### EXAMPLES

Examples 1-3 show processes to obtain amorphous olmesartan medoxomil. The olmesartan medoxomil obtained was characterized by X-Ray Powder Diffraction, DSC and TGA, and found to be amorphous olmesartan medoxomil having traces typified in FIGS. 1-3.

### Example 1

### Precipitation of Amorphous Olmesartan Medoxomil by Isothermal Evaporation of Solvent from a Methanol Solution of Olmesartan Medoxomil

Approximately 200 mg of olmesartan medoxomil was dissolved in 20 ml of HPLC-grade methanol. To achieve complete dissolution, the solution was exposed to ultrasonication for 5 minutes at room temperature. The resulting clear solution was filtered through a 0.45 μm filter, and the resulting clear filtrate was collected in a crystallisation dish. Full evaporation of methanol solvent was achieved at room temperature by leaving the open crystallisation dish in a fume-hood overnight. A coherent amorphous matrix was obtained, which was processed to a white amorphous powder.

### Example 2

### Precipitation of Amorphous Olmesartan Medoxomil by Isothermal Evaporation of Solvent from an Ethanol Solution of Olmesartan Medoxomil

Approximately 200 mg of olmesartan medoxomil was dispersed in 20 ml of anhydrous ethanol. To obtain a saturated solution, the dispersion was exposed to ultrasonication for 5 minutes at room temperature. The resulting dispersion was filtered through a 0.45 μm filter, and the resulting clear filtrate was collected in a crystallisation dish. Full evaporation of ethanol solvent was achieved at room temperature by leaving the open crystallisation dish in a fume-hood overnight. A coherent amorphous matrix was obtained, which was processed to a white amorphous powder.

### Example 3

### Precipitation of Amorphous Olmesartan Medoxomil by Isothermal Evaporation of Solvent from an Acetonitrile Solution of Olmesartan Medoxomil

Approximately 200 mg of olmesartan medoxomil was dispersed in 20 ml of HPLC-grade acetonitrile. To obtain a saturated solution, the dispersion was exposed to ultrasonication for 5 minutes at room temperature. The resulting dispersion was filtered through a 0.45 μm filter, and the resulting clear filtrate was collected in a crystallisation dish. Full evaporation of acetonitrile solvent was achieved at room temperature by leaving the open crystallisation dish in a fume-hood overnight. A coherent amorphous matrix was obtained, which was processed to a white amorphous powder.

1. Amorphous olmesartan medoxomil.

2. Amorphous olmesartan medoxomil characterized by an X-ray powder diffraction pattern substantially as shown in FIG. 1.

3. Amorphous olmesartan medoxomil characterized by a differential scanning calorimetry thermogram substantially as shown in FIG. 2.

4. Amorphous olmesartan medoxomil substantially free of water.

5. Amorphous olmesartan medoxomil according to claim 4, having a water content of less than about 2% by weight.

6. Amorphous olmesartan medoxomil substantially free of crystalline olmesartan medoxomil.

7. Amorphous olmesartan medoxomil according to claim 6, comprising less than about 10% crystalline olmesartan medoxomil.

8. Amorphous olmesartan medoxomil according to any one of claims 1 to 7, for use in medicine.

9. Amorphous olmesartan medoxomil according to any one of claims 1 to 8, for treating or preventing an angiotensin type II receptor mediated disorder.

10. Amorphous olmesartan medoxomil according to any one of claims 1 to 9, for treating or preventing hypertension.

11. A process for preparing amorphous olmesartan medoxomil, comprising the steps of:
(a) dissolving or suspending olmesartan medoxomil in one or more organic solvent(s); and
(b) isolating amorphous olmesartan medoxomil.

12. A process according to claim 11, wherein the solvent(s) is/are alcohol solvents or nitrile solvents.

13. A process according to claim 12, wherein the solvent(s) is/are short-chain alcohol solvents or short-chain nitrile solvents.

14. A process according to claim 13, wherein the solvent(s) is/are methanol, ethanol or acetonitrile.

15. A process according to claim 14, wherein the solvent(s) is/are anhydrous ethanol.

16. A process according to any one of claims 11 to 15, wherein the solvent(s) is/are HPLC-grade.

17. A process according to any one of claims 11 to 16, wherein the solution or suspension obtained in step (a) is subjected to sonication or heating to aid the dissolution of the olmesartan medoxomil.

18. A process according to any one of claims 11 to 17, wherein the solution or suspension obtained in step (a) is filtered.

19. A process according to claim 18, wherein the solution or suspension is filtered through a filter having a pore size of 0.3-1.0 μm.

20. A process according to claim 19, wherein the solution or suspension is filtered through a filter having a pore size of 0.4-0.6 μm.

21. A process according to claim 20, wherein the solution or suspension is filtered through a filter having a pore size of about 0.45 μm.

22. A process according to any one of claims 11 to 21, wherein the amorphous olmesartan medoxomil is isolated by allowing the solvent to evaporate.

23. A process according to claim 22, wherein the solvent is allowed to evaporate by spray drying, flash drying, heating, evaporation under reduced pressure, or evaporation under ambient conditions.

24. A process according to claim 23, wherein the solvent is allowed to evaporate under ambient conditions at a temperature of between 20-35° C.

25. A pharmaceutical composition comprising amorphous olmesartan medoxomil according to any one of claims 1 to 10, or prepared by a process according to any one of claims 11 to 24, and at least one pharmaceutically acceptable excipient.

26. A method of treating or preventing an angiotensin type II receptor mediated disorder, comprising administering to a subject in need of such treatment or prevention, a therapeutically or prophylactically effective amount of amorphous olmesartan medoxomil according to any one of claims 1 to 10, amorphous olmesartan medoxomil prepared by a process according to any one of claims 11 to 24, or a composition according to claim 25.

27. A method according to claim 26, wherein the disorder is hypertension.

28. Use of amorphous olmesartan medoxomil according to any one of claims 1 to 10, or use of amorphous olmesartan medoxomil prepared by a process according to any one of claims 11 to 24, or use of a composition according to claim 25, in the manufacture of a medicament for the treatment or prevention of an angiotensin type II receptor mediated disorder.

29. A use according to claim 28, wherein the disorder is hypertension.
